(19) United States
(12) Patent Application Publication
(10) Pub. No.: US 2024/0342391 A1
(43) Pub. Date: Oct. 17, 2024

(54) Needle Shield Remover with Cam Release

(71) Applicant: Becton, Dickinson and Company, Franklin Lakes, NJ (US)

(72) Inventor: Matthew C. Klimash, Highland Lakes, NJ (US)

(21) Appl. No.: 18/135,372

(22) Filed: Apr. 17, 2023

(51) Int. Cl. A61M 5/32; A61M 5/142; A61M 5/315

(52) U.S. Cl. CPC A61M 5/3204; A61M 5/14244; A61M 5/31576

(57) ABSTRACT

A needle shield remover for a wearable injector includes a first portion having a cam member, the first portion having a top surface and a bottom surface positioned opposite to the top surface, and a second portion connected to the first portion. The second portion having a top surface and a bottom surface positioned opposite to the top surface, with the second portion including a device connector configured to secure the second portion of the needle shield remover to the wearable injector. The first portion is moveable relative to the second portion between a first position where the cam member is aligned with the top surface of the second portion and a second position where the cam member protrude from the top surface of the second portion. The cam member is configured to contact a housing of the wearable injector when the first portion is in the second position.

### BACKGROUND OF THE INVENTION

### Field of the Invention

The present disclosure relates generally to a needle shield remover with a cam release member.

### Description of Related Art

Various types of automatic injection or drug delivery devices have been developed to allow drug solutions and other liquid therapeutic preparations to be administered by untrained personnel or to be self-injected. Generally, these devices include a reservoir that is pre-filled with the liquid therapeutic preparation, and some type of automatic needle-injection mechanism that can be triggered by the user. When the volume of fluid or drug to be administered is generally below a certain volume, such as 1 mL, an auto-injector is typically used, which typically has an injection time of about 10 to 15 seconds. When the volume of fluid or drug to be administered is above 1 mL, the injection time generally becomes longer, resulting in difficulties for the patient to maintain contact between the device and the target area of the patient's skin. Further, as the volume of drug to be administered becomes larger, increasing the time period for injection becomes desirable. The traditional method for a drug to be injected slowly into a patient is to initiate an IV and inject the drug into the patient's body slowly. Such a procedure is typically performed in a hospital or outpatient setting.

Certain devices allow for self-injection in a home setting and are capable of gradually injecting a liquid therapeutic preparation into the skin of a patient. In some cases, these devices are small enough (both in height and in overall size) to allow them to be “worn” by a patient while the liquid therapeutic preparation is being infused into the patient. These devices typically include a pump or other type of discharge mechanism to force the liquid therapeutic preparation to flow out of a reservoir and into the injection needle. Such devices also typically include a valve or flow control mechanism to cause the liquid therapeutic preparation to begin to flow at the proper time and a triggering mechanism to initiate the injection.

Referring to FIG. 16, in order to maintain sterility and to prevent accidental needle sticks, the needle of a drug delivery device is covered by a needle shield 2 prior to use. The needle shield 2 may be removed by grasping the needle shield or by grasping a needle shield remover 4 that removes the needle shield 2 when the needle shield remover 4 is separated from the device.

### SUMMARY OF THE INVENTION

In one aspect or embodiment, a needle shield remover for a wearable injector includes a first portion including a cam member, with the first portion having a top surface and a bottom surface positioned opposite to the top surface, and a second portion connected to the first portion, with the second portion having a top surface and a bottom surface positioned opposite to the top surface. The second portion includes a device connector configured to secure the second portion of the needle shield remover to the wearable injector, where the first portion is moveable relative to the second portion between a first position where the cam member is aligned with the top surface of the second portion and a second position where the cam member protrudes from the top surface of the second portion. The cam member is configured to contact a housing of the wearable injector when the first portion is in the second position.

The first portion may be connected to the second portion via a hinge. The hinge may be a living hinge. The first portion may be moveable from 0 to 90 degrees relative to the second portion. The cam member may be parallel to the top surfaces of the first portion and the second portion when the first portion is in the first position. The needle shield remover may include a needle shield including a body defining an opening configured to receive a needle of the wearable injector. At least one of the first portion and the second portion may include a needle shield surface configured to engage the needle shield. The first portion may include a handle.

In a further aspect or embodiment, a wearable injector includes a housing having a top surface and a bottom surface, a container received within the housing, with the container configured to receive a medicament and including a stopper configured to move within the container, a drive assembly configured to move the stopper, a needle having a retracted position where the needle is positioned within the housing and an extended position where a portion of the needle protrudes from the housing, and a needle shield remover according to any of the aspects or embodiments described above.

In a further aspect or embodiment, a method of using the needle shield remover of any of the aspects or embodiments described above includes: grasping the first position and moving the first portion from the first position to the second position; engaging a housing of the wearable injector with the cam member of the first portion; separating the needle shield remover from the housing the wearable injector; and removing a needle shield from the wearable injector.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a wearable injector according to one aspect of the present invention.

FIG. 2 is a perspective, cross-sectional view of the wearable injector of FIG. 1 according to one aspect of the present invention.

FIG. 3 is a front, cross-sectional view of the wearable injector of FIG. 1 according to one aspect of the present invention.

FIG. 4 is a top view of the wearable injector of FIG. 1 according to one aspect of the present invention, showing a top portion of the housing removed and the wearable injector in a pre-use position.

FIG. 5 is a top, cross-sectional view of the wearable injector of FIG. 1 according to one aspect of the present invention, showing the wearable injector in a pre-use position.

FIG. 6 is a front, cross-sectional view of the wearable injector of FIG. 1 according to one aspect of the present invention, showing the wearable injector in a pre-use position.

FIG. 7 is a top view of the wearable injector of FIG. 1 according to one aspect of the present invention, showing a top portion of the housing removed and the wearable injector in an initial actuation position.

FIG. 8 is a top, cross-sectional view of the wearable injector of FIG. 1 according to one aspect of the present invention, showing the wearable injector in an initial actuation position.

FIG. 9 is a front, cross-sectional view of the wearable injector of FIG. 1 according to one aspect of the present invention, showing the wearable injector in an initial actuation position.

FIG. 10 is a top view of the wearable injector of FIG. 1 according to one aspect of the present invention, showing a top portion of the housing removed and the wearable injector in a use position.

FIG. 11 is a top, cross-sectional view of the wearable injector of FIG. 1 according to one aspect of the present invention, showing the wearable injector in a use position.

FIG. 12 is a front, cross-sectional view of the wearable injector of FIG. 1 according to one aspect of the present invention, showing the wearable injector in a use position.

FIG. 13 is a top view of the wearable injector of FIG. 1 according to one aspect of the present invention, showing a top portion of the housing removed and the wearable injector in a post-use position.

FIG. 14 is a top, cross-sectional view of the wearable injector of FIG. 1 according to one aspect of the present invention, showing the wearable injector in a post-use position.

FIG. 15 is a front, cross-sectional view of the wearable injector of FIG. 1 according to one aspect of the present invention, showing the wearable injector in a post-use position.

FIG. 16 is a partial cross-sectional view of a prior art needle shield remover assembly.

FIG. 17 is a partial perspective view of a needle shield remover according to one aspect or embodiment of the present invention, showing a first position of the needle shield remover.

FIG. 18 is a partial perspective view of the needle shield remover of FIG. 17, showing movement from a first position to a second position of the needle shield remover.

FIG. 19 is a partial perspective view of the needle shield remover of FIG. 17, showing a second position of the needle shield remover.

FIG. 20 is a perspective view of the needle shield remover of FIG. 17, showing movement from a first position to a second position of the needle shield remover.

FIG. 21 is a perspective view of the needle shield remover of FIG. 17, showing a second position of the needle shield remover while partially engaged with a drug delivery device.

FIG. 22 is a perspective view of the needle shield remover of FIG. 17, showing a second position of the needle shield remover while disengaged from a drug delivery device.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

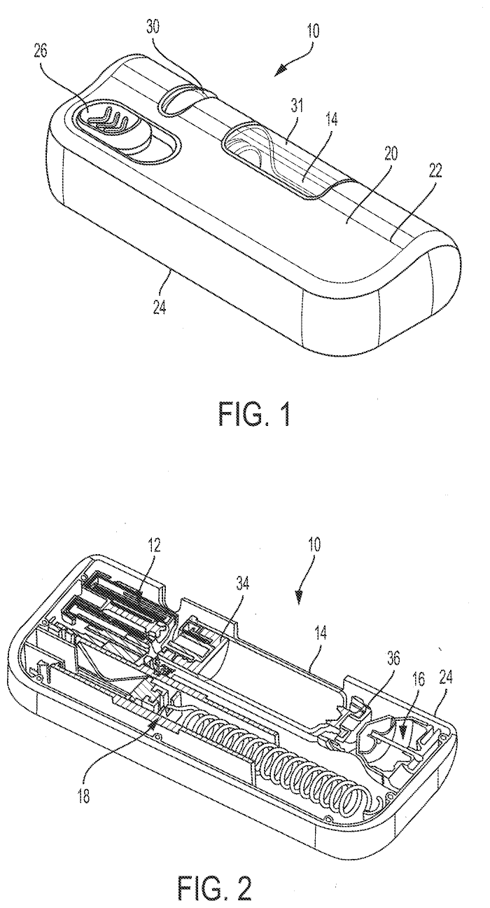

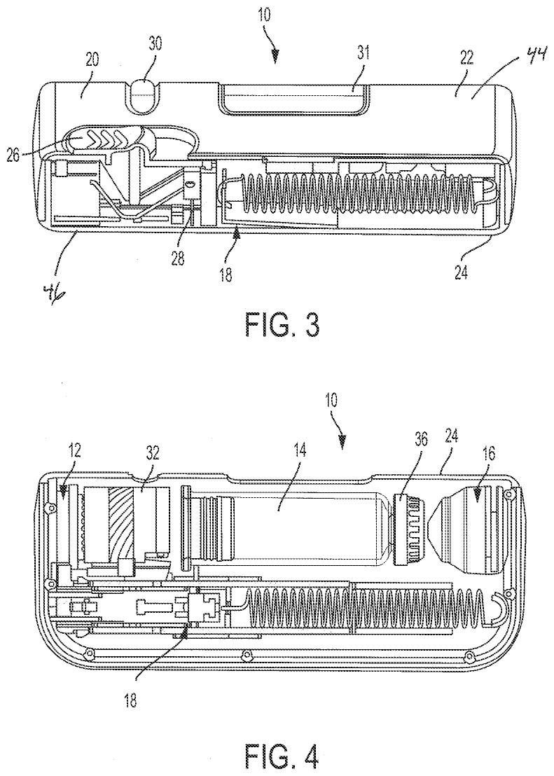

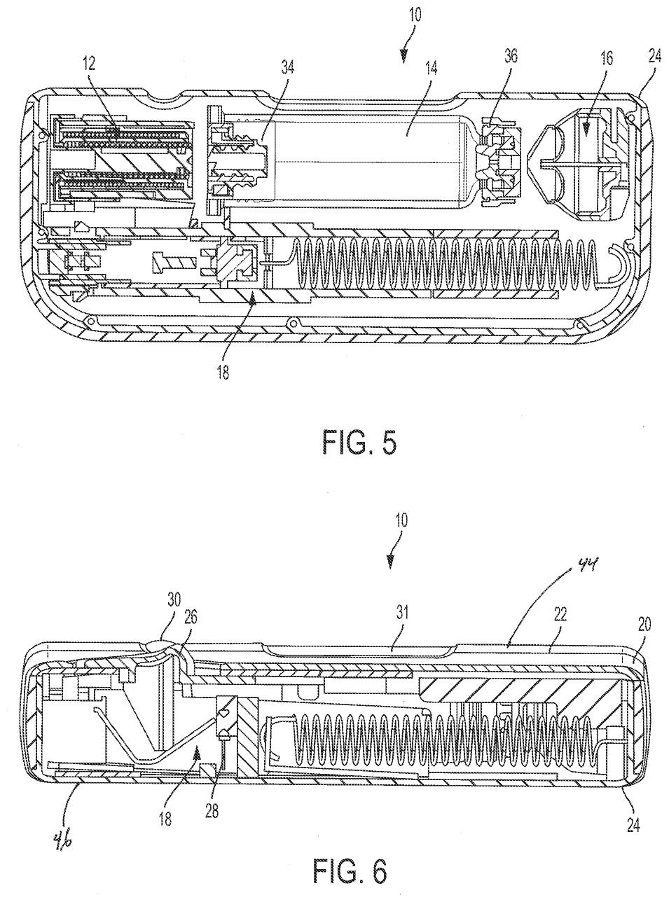

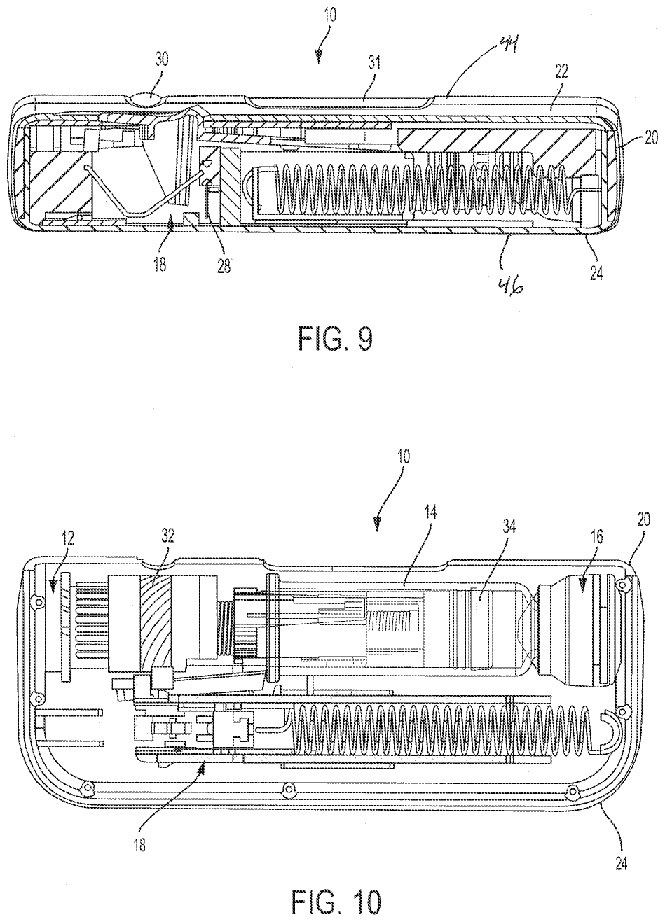

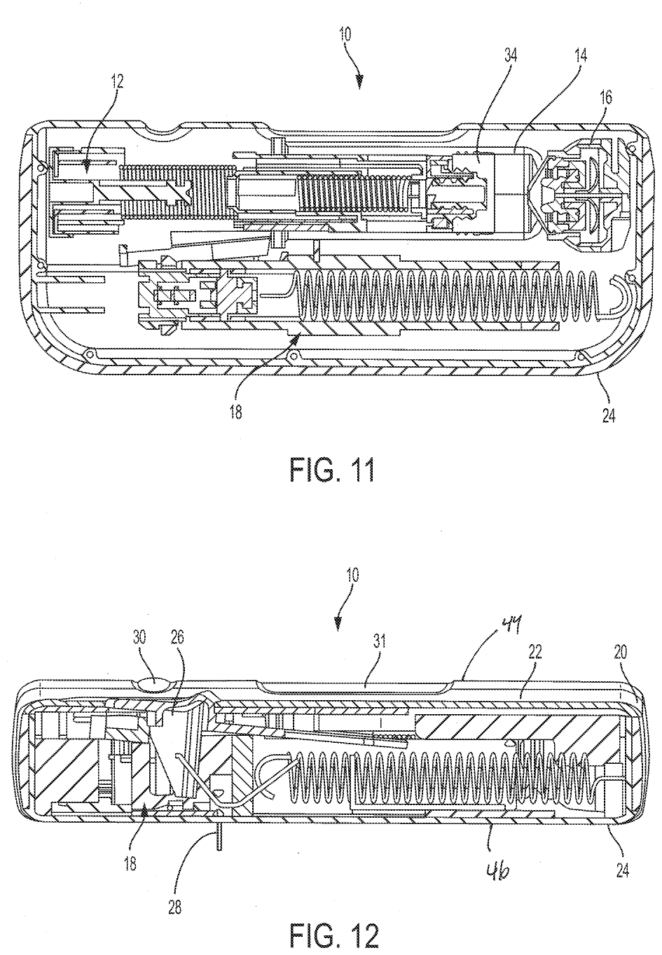

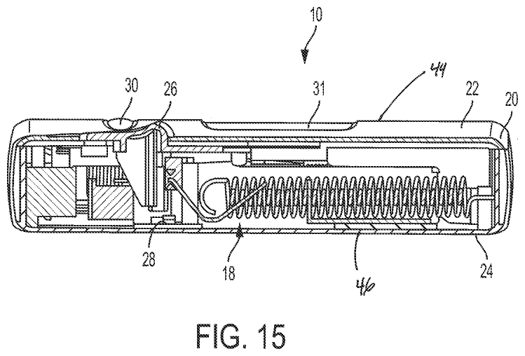

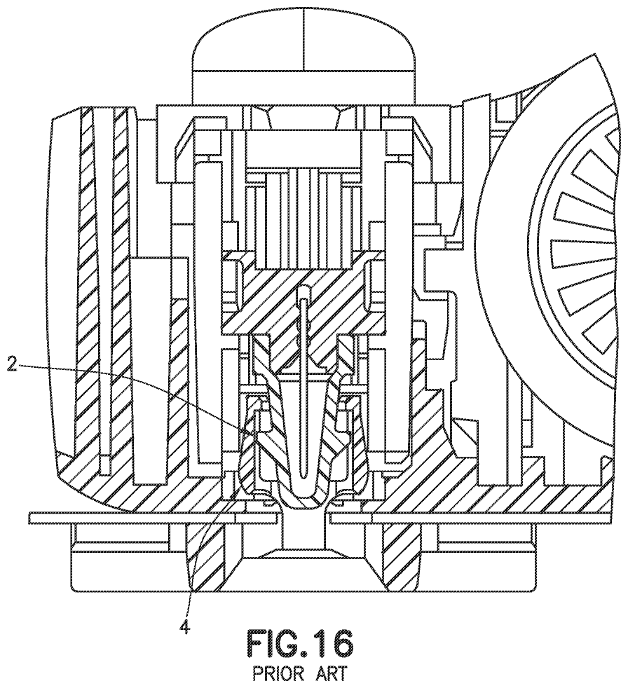

Referring to FIGS. 1-15, a wearable injector 10 according to one aspect of the present disclosure includes a drive assembly 12, a container 14, a valve assembly 16, and a needle actuator assembly 18. The drive assembly 12, the container 14, the valve assembly 16, and the needle actuator assembly 18 are at least partially positioned within a cavity defined by a housing 20. The housing 20 includes a top portion 22 and a bottom portion 24, although other suitable arrangements for the housing 20 may be utilized. In one aspect, the wearable injector 10 is an injector device configured to be worn or secured to a user and to deliver a predetermined dose of a medicament provided within the container 14 via injection into the user. The wearable injector 10 may be utilized to deliver a “bolus injection” where a medicament is delivered within a set time period. The medicament may be delivered over a time period of up to 45 minutes, although other suitable injection amounts and durations may be utilized. A bolus administration or delivery can be carried out with rate controlling or have no specific rate controlling. The wearable injector 10 may deliver the medicament at a fixed pressure to the user with the rate being variable. The general operation of the wearable injector 10 is described below in reference to FIGS. 1-15.

Referring again to FIGS. 1-15, the wearable injector 10 is configured to operate through the engagement of an actuation button 26 by a user, which results in a needle 28 of the needle actuator assembly 18 piercing the skin of a user, the actuation of the drive assembly 12 to place the needle 28 in fluid communication with the container 14 and to expel fluid or medicament from the container 14, and the withdrawal of the needle 28 after injection of the medicament is complete. The general operation of a wearable injector is shown and described in International Publication Nos. 2013/155153 and 2014/179774, which are hereby incorporated by reference in their entirety. The housing 20 of the wearable injector 10 includes an indicator window 30 for viewing an indicator arrangement 32 configured to provide an indication to a user on the status of the wearable injector 10 and a container window 31 for viewing the container 14. The indicator window 30 may be a magnifying lens for providing a clear view of the indicator arrangement 32. The indicator arrangement 32 moves along with the needle actuator assembly 18 during use of the wearable injector 10 to indicate a pre-use status, use status, and post-use status of the wearable injector 10. The indicator arrangement 32 provides visual indicia regarding the status, although other suitable indicia, such as auditory or tactile, may be provided as an alternative or additional indicia.

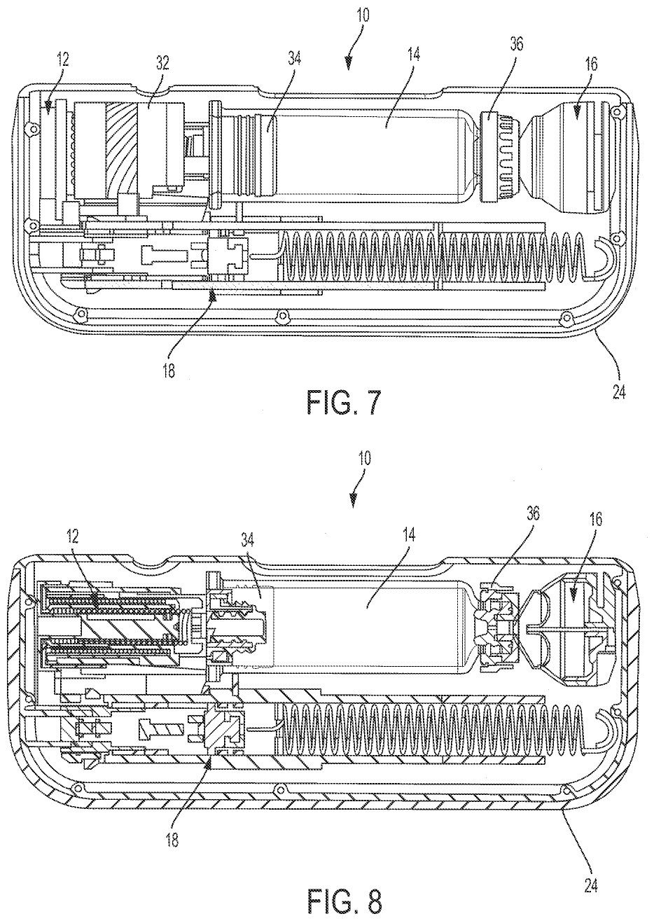

Referring to FIGS. 4-6, during a pre-use position of the wearable injector 10, the container 14 is spaced from the drive assembly 12 and the valve assembly 16 and the needle 28 is in a retracted position. During the initial actuation of the wearable injector 10, as shown in FIGS. 7-9, the drive assembly 12 engages the container 14 to move the container 14 toward the valve assembly 16, which is configured to pierce a closure 36 of the container 14 and place the medicament within the container 14 in fluid communication with the needle 28 via a tube (not shown) or other suitable arrangement. The drive assembly 12 is configured to engage a stopper 34 of the container 14, which will initially move the entire container 14 into engagement with the valve assembly 16 due to the incompressibility of the fluid or medicament within the container 14. The initial actuation of the wearable injector 10 is caused by engagement of the actuation button 26 by a user, which releases the needle actuator assembly 18 and the drive assembly 12 as discussed below in more detail. During the initial actuation, the needle 28 is still in the retracted position and about to move to the extended position to inject the user of the wearable injector 10.

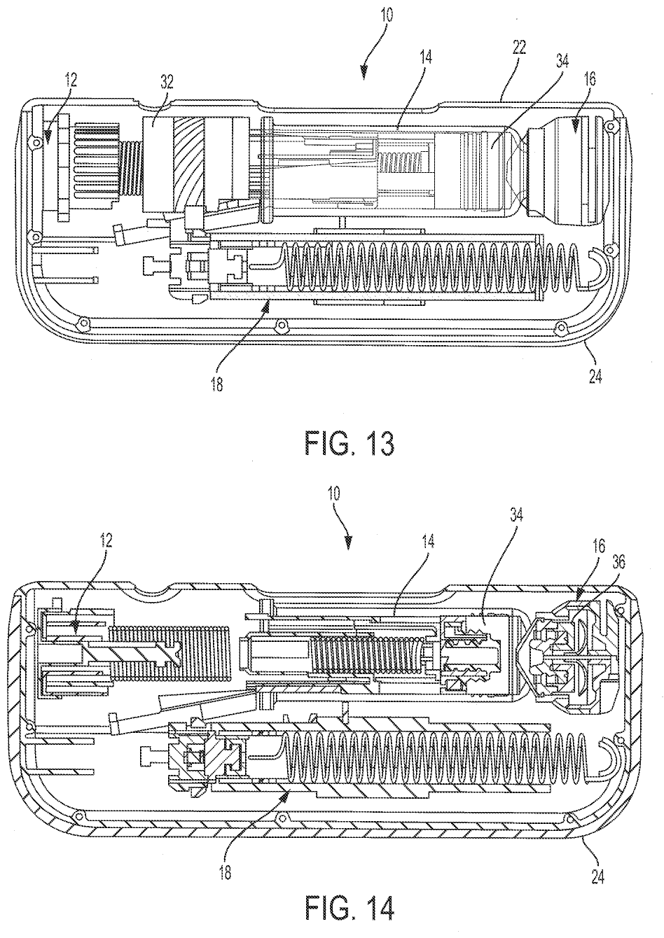

During the use position of the wearable injector 10, as shown in FIGS. 10-12, the needle 28 is in the extended position at least partially outside of the housing 20 with the drive assembly 12 moving the stopper 34 within the container 14 to deliver the medicament from the container 14, through the needle 28, and to the user. In the use position, the valve assembly 16 has already pierced a closure 36 of the container 14 to place the container 14 in fluid communication with the needle 28, which also allows the drive assembly 12 to move the stopper 34 relative to the container 14 since fluid is able to be dispensed from the container 14. At the post-use position of the wearable injector 10, shown in FIGS. 13-15, the needle 28 is in the retracted position and engaged with a pad 38 to seal the needle 28 and prevent any residual flow of fluid or medicament from the container 14. The container 14 and valve assembly 16 may be the container 14 and valve assembly 16 shown and described in International Publication No. WO 2015/081337, which is hereby incorporated by reference in its entirety.

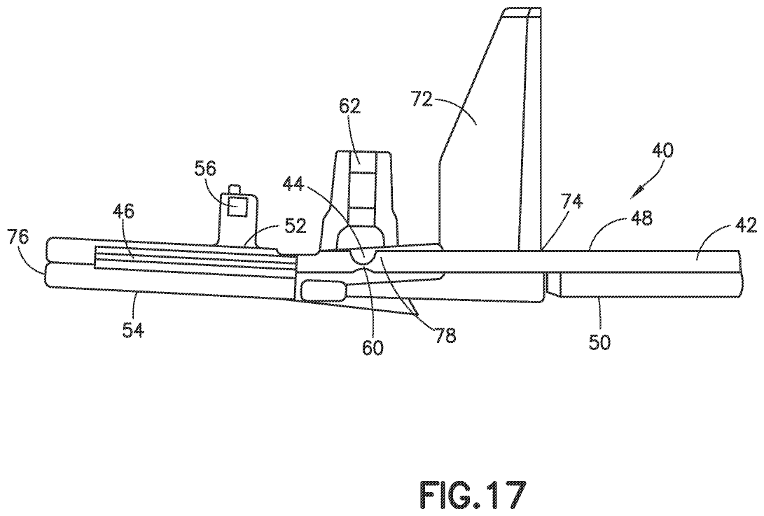

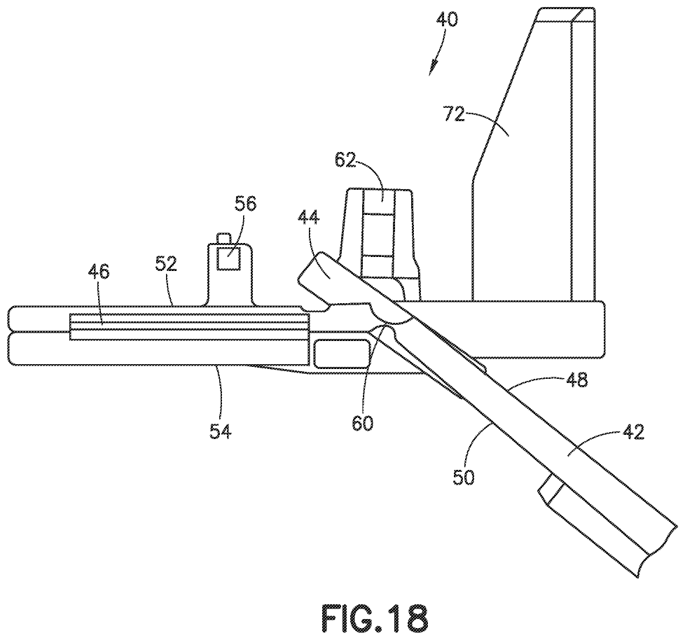

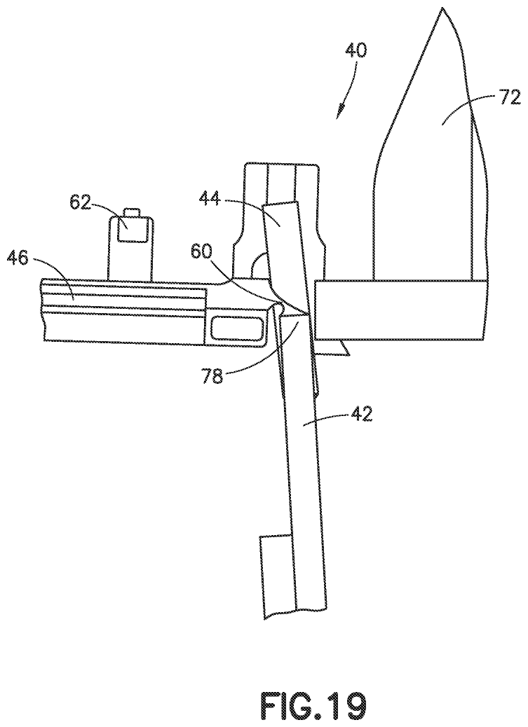

Referring to FIGS. 17-22, in one aspect or embodiment, the wearable injector 10 of FIGS. 1-15 further includes a needle shield remover 40 having a first portion 42 including a cam member 44 and a second portion 46 connected to the first portion 42. The first portion 42 having a top surface 48 and a bottom surface 50 positioned opposite to the top surface 48. The second portion 46 has a top surface 52 and a bottom surface 54 positioned opposite to the top surface 52. The second portion 46 includes a device connector 56 configured to secure the second portion 46 of the needle shield remover 40 to the wearable injector 10. The first portion 42 is moveable relative to the second portion 46 between a first position where the cam member 44 is aligned with the top surface 52 of the second portion 46 and a second position where the cam member 44 protrudes from the top surface 52 of the second portion 46. The cam member 44 is configured to contact the housing 20 of the wearable injector 10 when the first portion 42 is in the second position. As discussed in more detail below, separating the needle shield remover 40 from the wearable injector 10 also removes the needle shield 2 from the wearable injector 10. Movement of the first portion 42 from the first position to the second position is configured to cause the cam member 44 to engage the housing 20 of the wearable injector 10 to provide leverage and assist a patient or healthcare worker in separating the needle shield remover 40 and the needle shield 2 from the housing 20.

Referring again to FIGS. 17-22, in one aspect or embodiment, the first portion 42 is connected to the second portion 46 via a hinge 60. The hinge 60 may be a living hinge, although other suitable hinges and connection arrangements may be utilized. In one aspect or embodiment, the needle shield remover 40 includes two hinges 60 positioned on opposite sides of the needle shield remover 40 and two cam members 44 on opposite sides of the needle shield remover 40. However, the needle shield remover 40 may include one or more cam members 44 and one or more hinges 60. The first portion 42 may be moveable from 0 to 90 degrees relative to the second portion 46. As shown in FIG. 17, the cam member 44 is parallel to the top surfaces 48, 52 of the first portion 42 and the second portion 46 when the first portion 42 is in the first position. At least one of the first portion 42 and the second portion 46 includes a needle shield surface 62 configured to engage the needle shield 40. In one aspect or embodiment, as shown in FIGS. 17, 18, and 22, the second portion 46 of the needle shield remover 40 includes the needle shield surface 62. In some aspects or embodiments, the first portion 42 includes a handle 64. The handle 64 is configured to be grasped by a patient or healthcare worker to move the first portion 42 between the first and second positions. The handle 64 is circular, although other suitable arrangements may be utilized. The cam member 44 may be formed integrally with the first portion 42 or may be formed be separately from the first portion 42 and connected to the first portion 42. The cam member 44 may be formed from the same material as the first portion 42 or a different material than the first portion 42.

Referring to FIGS. 17-19, the needle shield remover 40 may also include one or more features, such as a protrusion 72, to prevent the actuation of the wearable injector 10 while the needle shield remover assembly 40 is connected to the wearable injector 10. The device connector 56, the needle shield surface 62, and the protrusion 72 extend from the top surface 52 of the second portion 46, although other suitable arrangements may be utilized. The hinge 60 is positioned between first and second ends 74, 76 of the second portion 46 and at a first end 78 of the first portion 42, with the first end 78 of the first portion 42 positioned opposite a second end 80. The cam member 44 extends beyond the first end 78 of the first portion 42, although other suitable arrangements may be utilized.

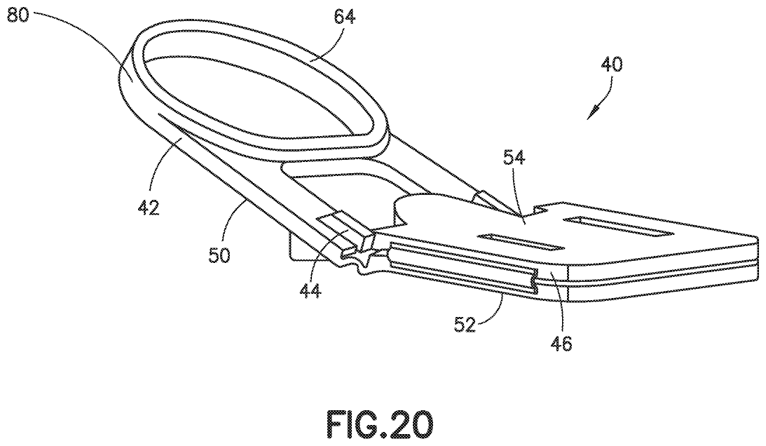

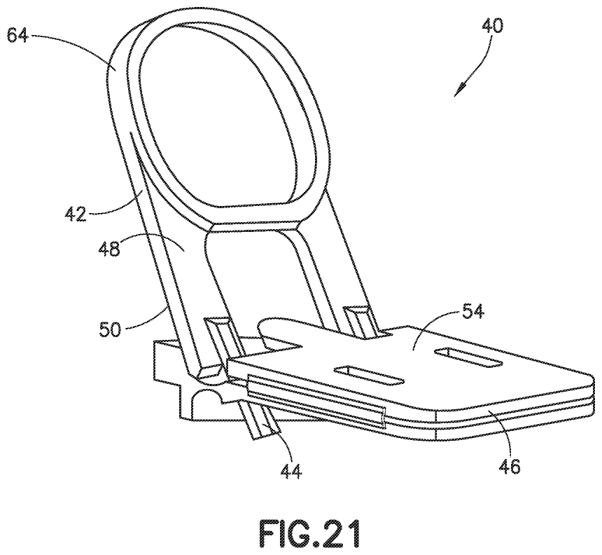

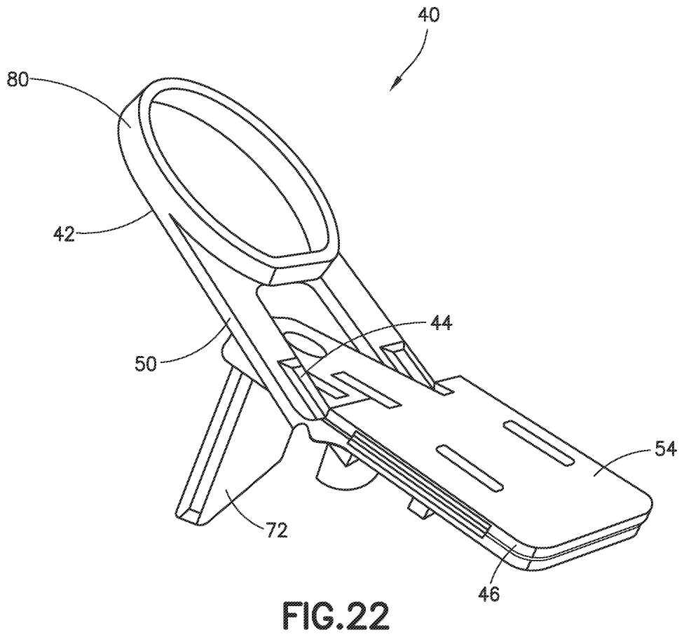

Referring to FIGS. 20-22, in one aspect or embodiment, the needle shield remover 40 is used by grasping the handle 64 and moving the first portion 42 of the needle shield remover 40 from the first position to the second position, as shown in FIG. 20, such that the first portion 42 moves relative to the second portion 46. The handle 64 is moved or pulled away from the housing 20 of the wearable injector 10. When the first portion 42 is moved to the second position, as shown in FIG. 21, the cam member 44 of the first portion 42 engages the housing 20 of the wearable injector 10 and provides a lever that assists in separating the needle shield remover 40 from the housing 20. Further pulling of the handle 64, as shown in FIG. 22, results in complete separation of the needle shield remover 40 from the housing 20. The needle shield remover 40 can be discarded and the wearable injector 10 can then be placed on a patient for delivery of a dose of medication.

Elements of one disclosed aspect can be combined with elements of one or more other disclosed aspects to form different combinations, all of which are considered to be within the scope of the present invention.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A needle shield remover for a wearable injector comprising:
a first portion comprising a cam member, the first portion having a top surface and a bottom surface positioned opposite to the top surface; and
a second portion connected to the first portion, the second portion having a top surface and a bottom surface positioned opposite to the top surface, the second portion comprising a device connector configured to secure the second portion of the needle shield remover to the wearable injector, wherein the first portion is moveable relative to the second portion between a first position where the cam member is aligned with the top surface of the second portion and a second position where the cam member protrudes from the top surface of the second portion, and wherein the cam member is configured to contact a housing of the wearable injector when the first portion is in the second position.

2. The needle shield remover of claim 1, wherein the first portion is connected to the second portion via a hinge.

3. The needle shield remover of claim 2, wherein the hinge comprises a living hinge.

4. The needle shield remover of claim 2, wherein the first portion is moveable from 0 to 90 degrees relative to the second portion.

5. The needle shield remover of claim 1, wherein the cam member is parallel to the top surfaces of the first portion and the second portion when the first portion is in the first position.

6. The needle shield remover of claim 1, further comprising a needle shield comprising a body defining an opening configured to receive a needle of the wearable injector.

7. The needle shield remover of claim 6, wherein at least one of the first portion and the second portion comprise a needle shield surface configured to engage the needle shield.

8. The needle shield remover of claim 1, wherein the first portion comprises a handle.

9. A wearable injector comprising:
a housing having a top surface and a bottom surface;
a container received within the housing, the container configured to receive a medicament, the container comprising a stopper configured to move within the container;
a drive assembly configured to move the stopper;
a needle having a retracted position where the needle is positioned within the housing and an extended position where a portion of the needle protrudes from the housing; and
a needle shield remover comprising:
a first portion comprising a cam member, the first portion having a top surface and a bottom surface positioned opposite to the top surface, and
a second portion connected to the first portion, the second portion having a top surface and a bottom surface positioned opposite to the top surface, the second portion comprising a device connector configured to secure the second portion of the needle shield remover to the wearable injector, wherein the first portion is moveable relative to the second portion between a first position where the cam member is aligned with the top surface of the second portion and a second position where the cam member protrudes from the top surface of the second portion, and wherein the cam member is configured to contact a housing of the wearable injector when the first portion is in the second position.

10. The wearable injector of claim 9, wherein the first portion is connected to the second portion via a hinge.

11. The wearable injector of claim 10, wherein the hinge comprises a living hinge.

12. The wearable injector of claim 10, wherein the first portion is moveable from 0 to 90 degrees relative to the second portion.

13. The wearable injector of claim 9, wherein the cam member is parallel to the top surfaces of the first portion and the second portion when the first portion is in the first position.

14. The wearable injector of claim 9, further comprising a needle shield comprising a body defining an opening configured to receive a needle of the wearable injector.

15. The wearable injector of claim 14, wherein at least one of the first portion and the second portion comprise a needle shield surface configured to engage the needle shield.

16. The wearable injector of claim 9, wherein the first portion comprises a handle.
